(19) United States
(12) Patent Application Publication
(10) Pub. No.: US 2005/0244897 A1
(43) Pub. Date: Nov. 3, 2005

(54) Endothelial cells as diagnostic instrument in cardiovascular diseases

(76) Inventors: Andreas M. Zeiher, Frankfurt am Main (DE); Stefanie Dimmeler, Frankfurt am Main (DE); Lothar Rossig, Frankfurt am Main (DE)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/534,150

(22) PCT Filed: Nov. 17, 2003
(86) PCT No.: PCT/EP03/12859

§ 371 Date: Jun. 29, 2005

(30) Foreign Application Priority Data
Nov. 16, 2002 (DE) 102-53-526.4

(51) Int. Cl. G01N 33/53; G01N 33/567

(52) U.S. Cl. 435/7.2

(57) ABSTRACT

The invention relates to a method for the immunocytological determination of apoptotic endothelial cells and/or endothelial precursor cells in samples such as, for example, peripheral blood, by means of, for example, throughflow cytometry or by means of a solid-phase immunoassay, as a diagnostic instrument in patients in whom a cardiovascular disease is either manifest or who carry the risk for the same and as an instrument by means of which the prevention and the therapeutic management of such a disease can be improved.

The invention relates to a method for the immunocytological determination of apoptotic endothelial cells and/or endothelial precursor cells in samples such as, for example, peripheral blood, by means of, for example, flow cytometry or by means of a solid-phase immunoassay, as a diagnostic instrument in patients, in which a cardiovascular disease is either manifest or who carry the risk for the same, and as an instrument by means of which the prevention and the therapeutic management of such a disease is improved.

Often, the diagnosis of a cardiovascular disease is initiated by the symptomatic manifestation of arteriosclerotic lesions, so-called plaques, which are brought out by an acute arterial occlusion. Such incidences, including myocardial infarction and stroke, do occur, if a prior existing arteriosclerotic plaque is destabilized and ablated, or even torn off. At the time of the manifestation of this disease, in most cases earlier stable states of arteriosclerosis had preceded for years, which not necessarily led to subjective disturbances or clinically noticeable symptoms. Established methods in order to identify patients with a starting or progredient vascular abnormity carrying a risk for the development of unstable plaques include the documentation of modifications of stress-ECG and ultrasonic examinations of the peripheral arteries. Nevertheless, these examinations detect only larger plaques that lead to a significant stenosis and obstruction of the blood flow, and generally only lead to clinical symptoms under physical stress.

In general, a damage to endothelial cells (ECs) is regarded to be a crucial event during the beginning of arteriosclerosis (Ross R. N. Engl. J. Med. 1999; 340: 115-126). The rate of renewal of ECs is increased in those regions of the vessels that are particularly sensitive for a later development of arteriosclerotic lesions (Caplan B. A., Schwartz C. J. Atherosclerosis. 1973; 17: 401-417). This suggests that numerous ECs die before the development of the lesions. Therefore, the programmed cellular death (apoptosis) can be regarded as one of the most important possible pathobiological incidences that are responsible for this. Indeed, pro-arteriogenic stimuli, belonging to which are traditional cardiovascular risk factors, such as oxidative stress caused by smoking or dysproteinemia, induce the apoptosis of EC in vitro. In contrast, the shear stresses that are caused by the laminar bloodflow have a markedly protective effect on the survival of ECs, in that the pro-apoptotic activation by arteriogenic stimuli is completely repressed (Gimbrone M. A., Nagel T., Topper J. N. J. Clin. Invest 1997; 99: 1809-1813; Dimmeler S., Haendeler J., Rippmann V., Nehls M., Zeiher A. M. FEBS Lett. 1996; 399: 71-74). In addition, those regions of the vessels that are particularly exposed to the laminar flow are not only characterized by a very low renewal rate of ECs (Davies P. F., Remuzzi A., Gordon E. J., Dewey C. F. Jr., Gimbrone M. A. Jr. T Proc. Natl. Acad. Sci. USA. 1986; 83: 2114-2117), these also rather seldom develop arteriosclerotic lesions (Caplan B. A., Schwartz C. J. Atherosclerosis. 1973; 17: 401-417). Such findings support the concept of a potential contribution of EC-apoptosis in the development of arteriosclerosis.

In addition to histopathological examinations using immunological experiments, in the past it was attempted to determine traces of an endothelial damaging. There were early reports about a significant increase of an endothelaemia in patients with myocardial infarction and severe angina lasting for several days (Hladovec J., Prerovsky I., Stanek V., Fabian J. Klin. Wochenschr. 1978; 56: 1033-1036). Concomitant with the development of a novel cytofluorescence-assay using immunomagnetic beads that are coupled to a monoclonal antibody (S-Endo-1), wherein the antibody is directed against cultured endothelial cells of the human umbilical vein (Human Umbilical Vein Endothelial Cells, HUVEC) (George F., Poncelet P., Laurant J. C., Massot O., Arnoux D., Lequeux N., Ambrosi P., Chicheportiche C., Sampol J. J. Immunol. Methods. 1991; 139: 65-75), floating ECs could be detected in the circulation of patients with Mediterranean spotted fever (Drancourt M., George F., Brouqui P., Sampol J., Raoult D. J Infect Dis. 1992; 166: 660-663) as well as in patients with an infection by cytomegalovirus (CMV) (Grefte A., van der Giessen M., van Son W., The T. H. J Infect Dis. 1993; 167: 270-277). Using an adapted immunocytological method being based on an antibody directed against an essentially identical native epitope (clone P1 H12), the concentration of circulating ECs in normal healthy adults was determined at 2.6±1.6/ml peripheral blood (Solovey A., Lin Y., Browne P., Choong S., Wayner E., Hebbel R. P. N. Engl. J. Med.; 1997: 337: 1584-1590). Measurements of microparticles, whose shedding by ECs could be a parameter for their apoptosis, followed by staining of EC-markers, in patients with acute cardiac syndrome (Acute Cardiac Syndrome) demonstrated increased microparticles of endothelial origin (Mallat Z., Hugel B., Ohan J., Leseche G., Freyssinet J. M., Tedgui A. Circulation. 1999; 99: 348-353). It was also shown that increased microparticles were found in patients suffering from a myocardial infarction. Although the determination of microparticles which could also be released following a pro-inflammatory activation of the endothelium does not allow for a discrimination between inflammatory activation and apoptosis of the ECs, a dependency from the activity of the disease was demonstrated (Mallat Z., Benamer H., Hugel B., Benessiano J., Steg P. G., Freyssinet J. M., Tedgui A. Circulation. 2000; 101: 841-843).

Earlier approaches in order to identify patients having a risk for the development of unstable arteriosclerotic lesions relied on biophysical functional determinations of the endothelium of vessels through measurements at the forearm or measurements of the coronary bloodflow, whereby it could be shown that these results indeed correlate with the later occurrence of cardiovascular incidences. This method, nevertheless, requires an arterial catheterization which drastically limits its use for the analysis of a future cardiovascular risk in an asymptomatic population. As a non-invasive alternative, a prognosis using different biochemical markers in samples of peripheral blood was proposed, due to the fact that an increase of the plasma-concentrations of these factors in the systemic circulation is associated with the activation and functional adverse effects of the endothelium. Due to the fact that pro-inflammatory processes indeed constitute the main mechanism being the basis of an endothelial activation and the progression of a cardiovascular disturbance, the pathogenesis of arteriosclerosis, identically to systemic heart insuffiency, exhibits characteristics that are shared with inflammatory phenomena of other diseases. Belonging to these are, in particular, the increases of pro-inflammatory cytokines and other plasma proteins. In consequence, the diagnostic specificity is lacking for all indicators of a vascular activation that are detectable in plasma that are currently available, and therefore these are only of limited clinical use for the stratification of a cardiovascular risk.

Therefore, most recent strategies are directed to measuring damaged ECs that are shedded by arteriosclerotic plaques as promising markers of vascular vulnerability. Using magnetic beads for the selective isolation of circulating endothelial cells in samples of peripheral blood of patients that suffered from coronary cardiac diseases of different phases, it was shown that an increase of the number of these cells in the circulation was connected with the presence of acute coronary symptoms (Dignat-George et al.). Nevertheless, due to methodological reasons, these authors could determine only a very small number of endothelial cells floating in the circulation, and they were not successful in characterising signs of an apoptotic cellular death of these cells.

Recently, the development of multicolor-flowcytometry has provided the bases as required for a more accurate analysis of cells that are circulating in the peripheral blood. Using this technology, the amount of circulating EC in healthy human test persons was found at 9.1/μ1. About 15% of those exhibited signs of an EC-activation, i.e. provided a positive result upon immunocytochemical staining of CD105 and CD106 (Mancuso P., Burlini A., Pruneri G., Goldhirsch A., Martinelli G., Bertolini F. Blood. 2001; 97: 3658-3661).

Most recent findings provide first evidence that an apoptosis of EC could also take place in vivo (Rossig L., Dimmeler S., Zeiher A. M. Basic Res. Cardiol. 2001; 96: 11-22). It was shown in an animal study that an increased apoptosis of ECs in the aortas and femural arteries of aged monkeys was associated with a dysfunction of the endothelium of vessels (Asai K., Kudej R. K., Shen Y. T., Yang G. P., Takagi G., Kudej A. B., Geng Y. J., Sato N., Nazareno J. B., Vatner D. E., Natividad F., Bishop S. P., Vatner S. F. Arterioscler. Thromb. Vasc. Biol. 2000; 20: 1493-1499). Such an endothelial dysfunction represents a generally accepted characteristic, pointing already during early phases of an arteriosclerosis to its later manifestation. As yet plainest indication for the appearance of EC-apoptosis in vivo, the histopathological analysis of arteriosclerotic plaques in the carotids of humans showed an EC-apoptosis in the downstream positioned fraction of the plaques (Tricot O., Mallat Z., Heymes C., Belmin J., Leseche G., Tedgui A. Circulation. 2000; 101: 2450-2453).

Also interesting are earlier insights into the pathomorphology of arteriosclerotic plaques that are connected with acute cardiac syndromes or sudden coronary death. These provide solid evidence for the fact that, in addition to a complete separation of the plaques, plaque-erosion to an essential extent additionally contributes to the triggering of the dramatic incidences (Virmani R., Kologdie F. D., Burke A. P., Farb A., Schwartz S. M. Arterioscler. Thromb Vasc. Biol. 2000; 20: 1262-1275). A plaque-erosion is characterized by a lack of endothelium, whereby only a minor inflammation of the exposed intima occurs (Farb A., Burke A. P., Tang A. L., Liang T. Y., Mannan P., Smialek J., Virmani R. Circulation. 1996; 93: 1354-1363). It is important, that a plaque-erosion is responsible for about 40% of the cases of thrombosis-induced sudden deaths (Farb A., Burke A. P., Tang A. L., Liang T. Y., Mannan P., Smialek J., Virmani R. Circulation. 1996; 93: 1354-1363). In addition, plaque-erosions are fairly common in diabetics or pre-menopausal women, and they increasingly occur also in persons with elevated CRP (C-reactive protein)-serum levels (Burke A. P., Tracy R. P., Kolodgie F., Malcom G. T., Zieske A., Kutys R., Pestaner J., Smialek J., Virmani R. Circulation. 2002; 105: 2019-2023).

Recent results have shown that the instability of plaque, and thus their potential to trigger a life threatening cardiovascular event, is not dependent from the extent of the plaques. Instead, unstable plaques that particularly tend to rip off or to erode are characterized by a destabilisation of the plaque-structure. The clinical pictures of an acute transformation of the vascular lesions go together with an increased EC-apoptosis, which constitutes a marker for the plaque instability. A pro-inflammatory activation and elevated apoptosis of the endothelium is also common in the non-arteriosclerotic caused heart failure. A quantification of the apoptosis by means of the analysis of endothelial cells that are shedded into the circulation (shedding) could ascertain the extent of the disease, before a clinical manifestation starts.

It is therefore an object of the present invention to develop a method, by which the risk to suffer from a cardiovascular adverse event that is related to uncontrolled modifications of the endothelial function can be estimated with the aid of an individual risk profile. This shall be achieved by the quantitative and critical determination of shedded, floating and circulating endothelial cells and/or endothelial precursor cells that constitute an indicator for the damaged endothelium. Suitable coutermeasures can then be undertaken by the attending physician based on the risk profile, in order to positively influence patients and to prevent the adverse event or at least to reduce it in its severity for the affected patient.

This object of the present invention is solved by a method for the identification and/or quantification of endothelial cells that are related to cardiovascular diseases in a sample. The method according to the invention comprises the steps of: (a) obtaining a sample to be analyzed containing endothelial cells; (b) incubating the sample with one or several molecules that specifically bind to one or several of the following marker molecules of the endothelial cells: i) endothelial cell-markers and/or markers for apoptosis, or ii) endothelial cell-markers and/or markers of endothelial precursor cells; (c) identification and/or quantification of the endothelial cells on the basis of the bound molecules by using immunocytological methods; and (d) comparing the result obtained for the sample to be analyzed with the result of a reference sample.

The results of the experiments that were performed in the context of the invention show that patients with coronary artery disease (Coronary Artery Disease, CAD) exhibit significantly elevated concentrations of circulating apoptotic ECs. These elevated values are strictly associated with the activity of the disease, since the highest concentrations were observed in patients with ACS. Accordingly, the amount of circulating apoptotic ECs could reflect the progression of a subclinical plaque erosion. In the experiments in the context of the invention, indeed elevated concentrations of apoptotic endothelial cells were observed in similar groups of patients, for which an increased occurrence of plaque erosion was reported.

Endothelial precursor cells are mobilized from the bone marrow in patients with myocardial infarction (Shintani S., Murohara T., Ikeda H., Ueno T., Honma T., Katoh A., Sasaki K., Shimada T., Oike Y., Imaizumi T. Circulation. 2001; 103: 2776-2779). Since recent experimental analyses have shown that endothelial precursor cells can contribute to a regeneration of the naked arterial sections (Walter D. H., Rittig K., Bahlmann F., Kirchmair R., Silver M. Murayama R., Nishimura H., Losordo D. W., Asahara T., Isner J. M. Circulation. 2002; 105(25): 3017-24.), the possibility exists that the pool of circulating endothelial precursor cells could represent an endogenous regenerative force following endothelial injury. Indeed the concentrations of endothelial precursor cells were about 2.3 fold increased in patients with ACS, compared to patients with stable CAD. Nevertheless, increased concentrations of apoptotic ECs are associated with significantly reduced concentrations of circulating endothelial precursor cells. These results can be interpreted in that a damaging of the vessel leads to a homing of the endothelial precursor cells, whereby the overall pool of circulating EC is reduced. On the other hand, the reduction of circulating endothelial precursor cells that is observed in patients with CAD (Vasa M., Fichtlscherer S., Aicher A., Adler K., Urbich C., Martin H., Zeiher A. M., Dimmeler S. Circ. Res. 2001; 89: E1-7) that is accompanied by an elevated EC-apoptosis could limit the regeneration of the endothelium by precursor cells and thus additionally affect the functional integrity of the endothelium-monolayer. Additional experimental examinations in animal models are required in order to understand these complex processes.

Preferred is a method according to the invention, wherein the endothelial cells are derived from a mammal, in particular from a human. Further preferred is a method according to the invention, wherein the endothelial cells to be analyzed or to be identified and/or quantified are selected from the group consisting of apoptotic endothelial cells, endothelial precursor cells and mature endothelial cells.

According to a further aspect of the method according to the invention, the sample to be analyzed can be any sample containing endothelial cells. The sample can be pre-treated, whereby e.g. a coagulation inhibitor, in particular heparin, can be added to peripheral blood, furthermore, a lysis of the erythrocytes between step (a) and (b) can be performed, or the sample can be used directly. Preferred is a method according to the invention, wherein the sample to be analyzed is selected from the group consisting of peripheral blood, cellular culture-suspensions and suspensions containing cells that have been mechanically, chemically and/or enzymatically released from the wall of the vessel. Such enzymatic release can be achieved by, e.g., collagenase. Particularly preferred is a method according to the invention, wherein the sample to be analyzed is peripheral blood.

An essential aspect of the method according to the invention is the incubation of the sample with one or several molecules that specifically bind to one or several of the marker molecules of the endothelial cells. These molecules can be selected from a very large multitude of molecules that are specific for endothelial cells. It is preferred that the marker-binding molecules are selected from the group consisting of antibodies or parts or fragments thereof, and receptor ligands or parts thereof. In particular, a very large number of peptides, proteins, and smaller molecules can be considered as receptor ligands, such as hormones and the like. These specifically bound molecules are then used as a basis for the further analysis, identification and/or quantification of the endothelial cells. Particularly preferred is a method according to the invention, wherein the antibodies or parts or fragments thereof comprise polyclonal antibodies, monoclonal antibodies, Fab-fragments, single chain-antibodies, and diabodies.

According to a further aspect of the method of the present invention, components of the method can be present bound to a solid phase, thus, the marker-binding molecules can be present in solution or matrix-immobilised. A multitude of materials that are known to the person of skill can be used as matrices, such as, for example, resin-matrices and/or common column matrices. Furthermore, particularly preferred is a method according to the invention, wherein the marker-binding molecules are coupled to one or several detection molecules from the group consisting of fluorescein thioisocyanate, phycoerythrine, enzymes (for example horseradish-peroxidase), and magnetic beads.

According to a further aspect of the method according to the invention, the marker-binding molecules can be detected with an antibody to which one or several detection molecules are coupled. This therefore represents an indirect detection of the binding of the molecule to the respective endothelial cellular marker. Such two-stepped determinations are very well known to the person of skill, such as, for example, from anti-antibody-detection technology.

An essential aspect of the method according to the invention are the endothelial cellular markers themselves to which the above mentioned molecules bind. These markers can be selected from all markers that are specific for endothelial cells. It is preferred that the endothelial cellular marker is selected from the group consisting of CD146, von Willebrandt-factor (vWF), and vascular endothelial growth factor-receptor 1 (VEGF-receptor-1); the apoptosis-marker is selected from the group consisting of annexin V, and PD-ECGF, and the markers of endothelial precursor cells are selected from the group consisting of CD133 and CD34. Nevertheless, these are only examples for markers starting from which the person of skill can readily determine and employ additional ones.

According to a further aspect of the method according to the invention, furthermore at least one marker that is characteristic for non-endothelial cells can be detected, such as, for example, CD45. This serves for a negative separation of the endothelial cells to be identified and/or quantified from other cells that are contained in the sample.

According to a further aspect of the method according to the present invention immunocytological methods can be employed for the identification and/or quantification of the endothelial cells. For this, all methods are suitable that allow for a specific determination based on the marker/molecule-interaction. Preferred are methods that are selected from the group consisting of flow cytometry and solid-phase-immunoassays. Thereby, also so-called “cell-sorters” can be used.

The ascertained data of the determination(s) of the endothelial cells from the sample are usually compared with a reference sample. What sample can be used as a reference sample will in particular depend from the kind of the sample to be examined, and the history of the disease of the individual from which the sample to be analysed is derived. Preferred is a method according to the invention, wherein the reference sample are derived from one or the mean value of several mammals, wherein a cardiovascular disease was excluded. Nevertheless, this is not mandatory if, e.g., the progression of a disease shall be determined also an “old” sample of the same patient can be used as a reference sample. It will be obvious for the person of skill, which samples are suitable as reference sample for the method according to the invention.

For an analysis of the results as obtained by the method according to the invention, for example, the result for apoptotic endothelial cells is brought in relation with the result for the totality of endothelial cells, and/or the result for apoptotic endothelial cells is brought in relation with the result for the endothelial cells.

According to a further aspect of the method according to the present invention, the cardiovascular diseases that are to be diagnosed and/or prognosed and/or whose therapy is to be monitored can be selected from the group consisting of stable and unstable angina, myocardial infarction, acute cardiac syndrome, coronary arterial disease and heart insufficiency. Nevertheless, it shall not be excluded that the method according to the invention can be used for further cardiologic disease states.

The invention preferably provides a flow-cytometric method for the determination of shedded, circulating EC in peripheral blood. A sensitive and highly specific novel method is provided by which the damaged endothelium preceding a plaque development can be detected, and the transition from a stable to an unstable arteriosclerotic phenotype can be quantified. The method not only allows for a higher sensitivity, compared to earlier methods, but also offers the possibility to further characterize the cells in view of their survival and their state of differentiation and their origin within the vascular system. Such a detailed profile regarding the amount and properties of apoptotic circulating ECs provides information with respect to the state of the endothelium that is superior to all markers that are currently available. The method of flow cytometry can also be adapted for the use as a solid phase immunoassay (solid-state immunosorbant assay), whereby a simplified practical use of the same principle is provided.

A further aspect of the method according to the invention relates to a diagnostic kit, wherein said kit comprises means for performing the method according to the invention, optionally together with additional components and/or excipients. Such means are preferably at least one antibody for determining of at least one endothelial marker, and means for the subsequent identification and/or quantification of the endothelial cells. Furthermore, the kit can contain other components and/or enzymes for performing the method of the present invention, e.g. manuals for the interpretation of the results of the assay regarding the risk profile of the patient, and corresponding countermeasures and proposals for therapy.

In summary, the results of the study that forms the basis of the invention show that patients with ACS (acute myocardial infarction and unstable angina) exhibit a threefold increase of circulating apoptotic ECs. Hypercholesterolemia, diabetes, and increased CRP-serum levels significantly correlate with increased concentrations of apoptotic ECs. The EC-apoptosis was significantly reduced by a four-week treatment of the patients with HMG-CoA-reductase-inhibitors. In total, these data suggest that a determination of circulating apoptotic ECs provides method for the monitoring of the activity of arteriosclerotic diseases in humans that, until now, was not available. Thus, an analysis of the cardiovascular risks as well as the primary diagnosis of an arteriosclerotic vascular disease together with the prognostic stratification of patients with manifest cardiovascular disease, including coronary, peripheral, and cerebral arterial diseases and progressive congestive heart failure, becomes possible.

A further aspect of the present invention thus relates to the use of the method according to the invention for the diagnosis and/or prognosis of cardiovascular diseases, and/or for monitoring of their therapy. This takes place by the quantitative and critical determination of shedded, floating and circulating endothelial cells and/or endothelial precursor cells as an indicator for a damaged endothelium. Based on the risk profile that can be produced thereupon, suitable countermeasures can then be initiated by the attending physician in order to positively influence the patient, and to prevent the adverse incidence or at least to reduce it in its severity for the affected patient. Such a therapy according to the invention can comprise the administration of lipid lowering substances that are selected from the group consisting of statines, in particular atorvastatin. Nevertheless, further possible therapies are known to the person of skill in order to treat cardiovascular diseases that can be performed according to common schemes.

Furthermore, the results of the studies that have been performed in the context of the invention for the first time elucidate the effects of classical risk factors for CAD on the apoptosis of ECs. A key role of lipids—in particular oxidised lipids—as triggers for EC-apoptosis has been proposed by different in vitro cell culture experiments (Dimmeler, S., Haendeler J, Galle J., Zeiher A. M. Circulation. 1997; 95: 1760-1763; Harada-Shiba M., Kinoshita M., Kamido H., Shimokado K. J. Biol. Chem. 1998; 273: 9681-6987). In addition, the results of the present study show that LDL-serum concentrations significantly correlate with an increased EC-apoptosis in patients with CAD. It is important that a treatment with the lipid lowering substance atorvastatin, an HMG-CoA-reductase-inhibitor, within a four week treatment period profoundly reduces the EC-apoptosis. Furthermore, a diabetic disease was associated with an increased EC-apoptosis. This could be explained based on results from in vitro-studies that show that high glucose concentrations induce the EC-apoptosis (Baumgartner-Parzer S. M., Wagner L., Pettermann M., Grillari J., Gessl A., Waldhausl W. Diabetes. 1995; 44: 1323-1327; Ho F. M., Liu S. H., Liau C. S., Huang P. J., Lin-Shiau S. Y. Circulation. 2000; 101: 2618-2624).

In addition, patients with diabetes are characterised by an increased oxidative stress, one of the most effective triggers of EC-apoptosis in vitro (Rössig L., Dimmeler S., Zeiher A. M. Basic. Res. Cardiol. 2001; 96: 11-22). Finally, the results as presented here also point to a significant connection between a low-grade inflammation and EC-apoptosis, in particular due to the finding that serum levels of the C-reactive protein (CRP), an established inflammatory marker, and indeed not only in patients with CAD but also in healthy control persons, are significantly associated with EC-apoptosis. Results from cell culture experiments have shown that mediators of inflammation efficiently stimulate an EC-apoptosis (Robaye B., Mosselmans R., Fiers W., Dumont J. E., Galand P. Am. J. Pathol. 1991; 138: 447-453). On the other hand, CRP-serum concentrations proved to be reliable predictive factors for the progression of an arteriosclerotic disease, being independent from classical CAD-risk factors (Blake G. J., Ridker P. M. Circ. Res. 2001; 89: 763-771). In summary, the significant connection between apoptotic ECs and CAD-risk factors further strengthened the concept that the numbers of circulating apoptotic EC indeed are associated with the activity of an arteriosclerotic disease.

In the following, the invention shall now be described in more detail based on the examples with respect to the accompanying Figures, without being limited thereto. The Figures show:

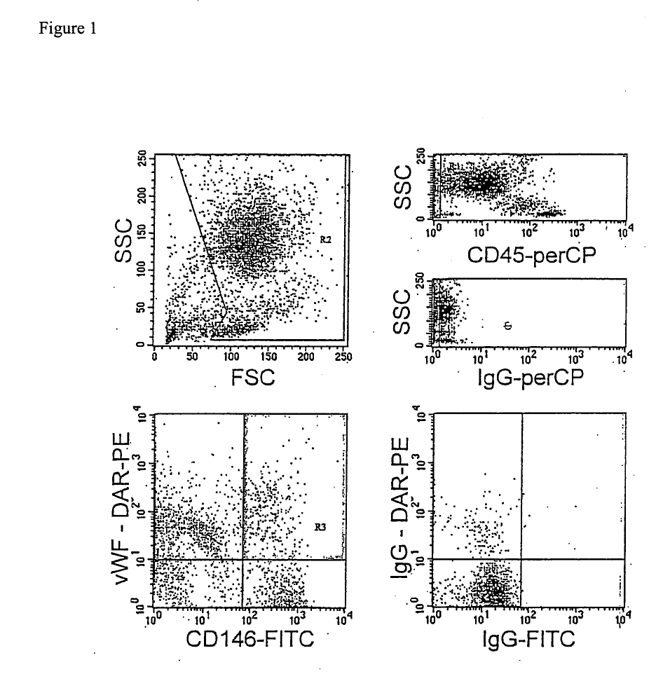

FIG. 1: Flow cytometry measurement of circulating ECs.

Circulating cells from peripheral blood were analysed after lysis of the erythrocytes. Upper left: In the illustration of the forward/sideward-scattering (Forward Scatter, FSC/Sideward Scatter, SSC), platelets and debris were masked for the further analysis (region R2). Upper right: anti-CD45-perCP-staining (channel FL-3, top, vs. isotype-control, bottom) for identifying circulating cells that are no leukocytes for the further analysis (region R1, blue). Bottom left: CD45-cells were selected for a quantification of CD146-(FITC-conjugated, channel FL-1) and vWF-(stained with a PE-labelled secondary antibody, channel FL-2) double positive cells (yellow marker). Bottom right: false-positive signals after staining with isotype control antibodies were subtracted from the original data, and the resulting EC-number was normalised with reference to the number of the white blood cells (WBC).

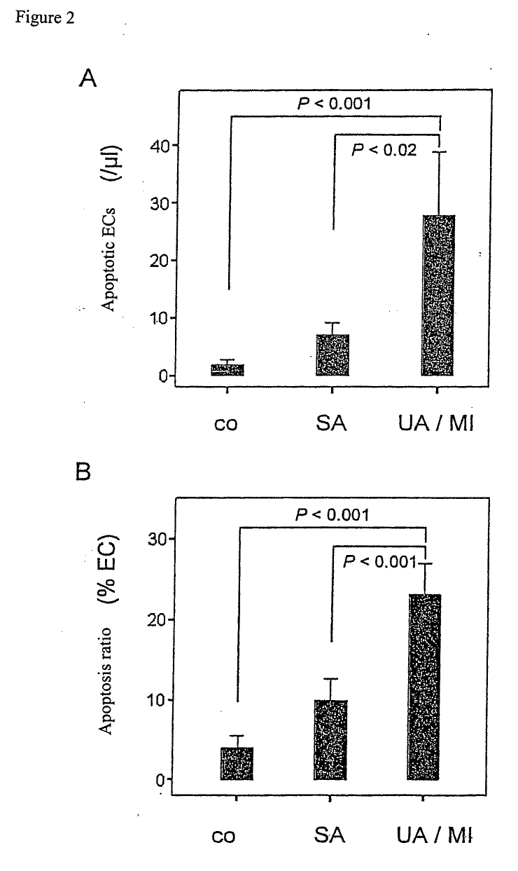

FIG. 2: Number of circulating apoptotic ECs (A), and relative ratio related to total circulating ECs (B), in healthy, age-matched control persons (n=41), patients with stable angina (SA; n=29), and patients with ACS (UA, unstable angina, and MI, myocardial infarction; n=27).

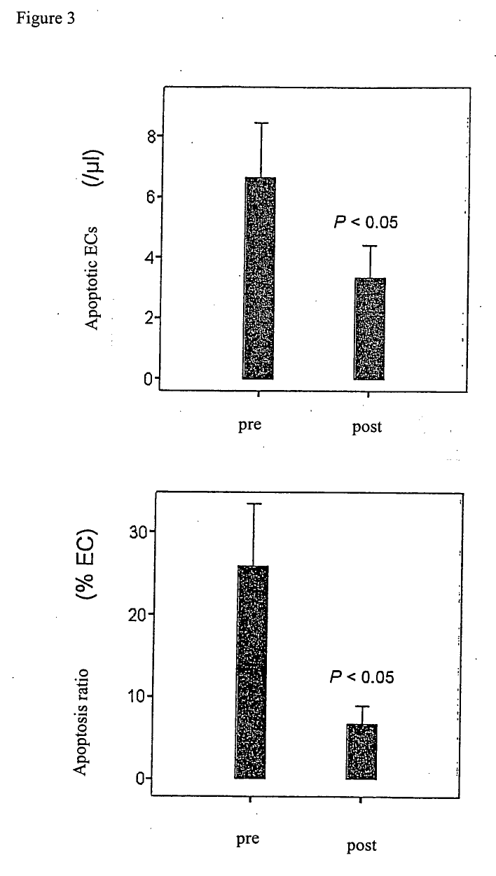

FIG. 3: Effect of a prospective treatment with atorvastatin (20 mg/day, 4 weeks) in n=8 patients with SA on the number of circulating apoptotic ECs (A), and on the portion of the apoptotic ECs in total circulating ECs (B).

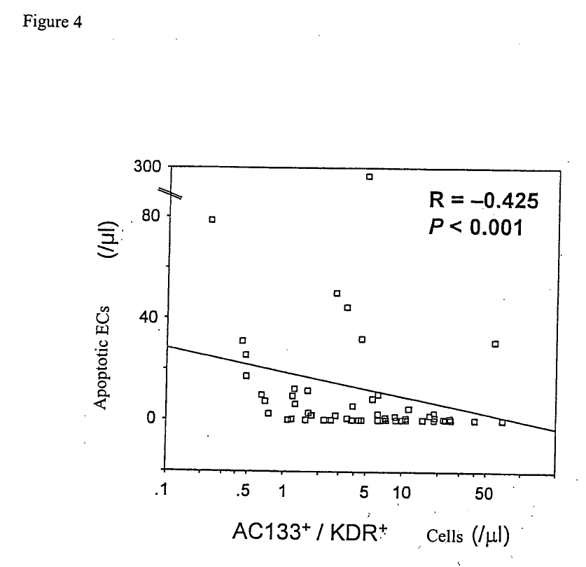

FIG. 4: Ratio between circulating apoptotic ECs and AC133+/KDR+endothelial precursor cells in patients with CAD.

### EXAMPLES

Material and Methods

1. Patients

In total 56 patients were examined. 29 patients suffered from stable angina (SA) for three months, defined as angiographically documented coronary arterial disease and stability of the angina under stress, before the blood samples were obtained. 27 patients were examined with unstable angina (unstable angina, UA), defined as angina pectoris de novo, angina pectoris crescendo or angina pectoris under resting conditions (14 patients), or in which within the last 7 days an ACS (troponin T-positive without enhancement of the ST-stretch) or an acute myocardial infarction (MI), together with an ST-enhancement, occurred (13 patients). Exclusion criteria were clinical or biochemical indications of an accompanying inflammatory disease, chronic renal insufficiency or a malign disease. In all patients, a coronary arterial disease was detected, wherein the inducing lesion was identified by coronary angiography. The clinical characteristics of these patients are summarized in Table 1, below. 41 healthy individuals, in which no indication for CAD could be found based on their anamnesis or based on a physical examination and which were matched in age to the patients, served as a control group.

TABLE 1

| Factor | Control | SA | UA/MI |
|---|---|---|---|
| n | 41 | 29 | 27 |
| Age | 58 ± 1 | 60 ± 2 | 61 ± 2 |
| Gender (m/f) | 30/11 | 23/6 | 19/8 |
| HTN (%) | — | 19 (66) | 20 (74) |
| Diabetes mellitus | — | 7 (24) | 10 (37) |
| (%) | | | |
| Nicotine (%) | — | 17 (59) | 15 (56) |
| Family history (%) | — | 17 (59) | 12 (44) |
| LDL-cholesterol | 132 ± 6 | 123 ± 7 | 134 ± |
| (mg/dl) | | | |
| HDL/LDL-ratio | 0.47 ± 0.04 | 0.43 ± 0.04 | 0.32 ± 0.02 |
| CRP (mg/dl) | 0.47 ± 0.06 | 0.65 ± 0.09 | 2.00 ± 0.35 |
| x-vessel | — | 2.00 ± 0.27 | 1.96 ± 0.17 |
| LVEF (%) | — | 49.78 ± 3.07 | 48.83 ± 2.67 |

In addition, 8 patients with stable angina were prospectively treated with atorvastatin (20 mg/day, four weeks). The clinical characteristics of these patients are summarized in Table 2, below.

TABLE 2

| | |
|---|---|
| n | 8 |
| Age | 66 ± 4 |
| Gender (m/f) | 30/11 |
| HTN (%) | 5 (63) |
| Diabetes mellitus | 2 (25) |
| (%) | |
| Nicotine (%) | 5 (63) |
| Family history (%) | 4 (50) |
| LDL-cholesterol | 155 ± 16 |
| (mg/dl) pre | |
| LDL-cholesterol | 86 ± 13 |
| (mg/dl) post | |
| CRP (mg/dl) | 1.53 ± 0.58 |
| x-vessel | 2.38 ± 0.32 |
| LVEF (%) | 52.9 ± 5.9 |

All patients and control persons had given their written consent. The study was approved by the ethics commission of the Johann Wolfgang von Goethe-Universitat, Frankfurt/Main.

In all patients serum was also collected at the time of examination for a determination of the concentrations of CRP and hsCRP (turbidimetric assay, Boehringer Mannheim and Ultrasensitive N Latex CRP Monotest, Behring, respectively) as well as serum lipid-fractions (Boehringer Mannheim).

2. Flow Cytometry

The lysis of erythrocytes in 10 ml peripheral venous heparin blood was performed with the aid of a commercially available solution for lysis (Becton Dickinson, BD). The circulating ECs were then examined by means of 4-channel-FACS (Fluorescence Activated Cell Sorting)-analysis, using a suitably modified protocol according to the method of Mancuso et al. and Monestiroli et al. (Mancuso P., Burlini A., Pruneri G., Goldhirsch A., Martinelli G., Bertolini F. Blood. 2001; 97: 3658-3661; Monestiroli S., Mancuso P., Burlini A., Pruneri G., Dell'Agnola C., Gobbi A., Martinelli G., Bertolini F. Cancer Res. 2001; 61: 4341-4344) (FIG. 1). First, in the representation of the forward/sideward-scatter a regional window was defined, in order to exclude platelets and debris from the further analyses. Amongst the remaining cells, the number of the CD45⁺-signals that were identified with a direct perCP-conjugated monoclonal antibody against human CD45 (BD) was quantified, in order to normalise each measured cellular population in view of the total number of the leukocytes in relation to the number of WBCs. In order to specifically detect mature ECs, only CD45⁻-cells were analysed further by double staining with antibodies against the EC-specific epitope CD146 (MeI-CAM, MUC18, S-Endo-1; Shih I. M. J Pathol. 1999; 189: 4-11) (monoclonal mouse-antibody, directly FITC-conjugated; Chemicon) and against von Willebrandt-factor (vWF) (rabbit, Oncogene), followed by a PE-conjugated anti-rabbit secondary antibody. Alternatively, human KDR was detected with an anti-KDR-antibody of the mouse (Sigma), followed by a PE-conjugated anti-mouse secondary antibody in addition to vWF. CD45⁻/CD146⁺/vWF⁺ or CD45⁻/KDR⁺/vWF⁺-cells were defined as mature ECs. These cellular populations were examined with respect to annexin V-binding by means of incubation with primary annexin V-APC-conjugates (Bender Medical Systems) (van England M., Nieland L. J., Ramaekers F. C., Schutte B., Reutelingsperger C. P. Cytometry. 1998; 31: 1-9). Isotype-identical antibodies served as controls (IgG1-PE and IgG2α-FITC, BD), and the annexin-binding was confirmed by incubation with streptavidin-coupled APCs or with isotype-specific APCs. A staining after incubation with isotype-control antibodies was regarded as false-positive, and subtracted from the original data (FIG. 1, bottom right). Each analysis includes 100,000 signals.

The detection of endothelial precursor cells was performed in a subgroup of the patients, as already described earlier (Vasa M., Fichtlscherer S., Aicher A., Adler K., Urbich C., Martin H., Zeiher A. M., Dimmeler S. Circ. Res. 2001; 89: E1-7). 100 μl of peripheral blood were incubated with FITC- or perCP-conjugated monoclonal antibodies against human CD34 (BD). As a set of secondary markers that recognize immature precursor cells, blood samples were immunostained with monoclonal antibodies against human CD133 (Milteny; PE-conjugated), and against human KDR (Sigma), followed by an FITC-conjugated secondary antibody. Isotype-identical antibodies served as controls (BD). Following the incubation, the cells were lysed, washed with PBS and fixed in 4% para-formaldehyde, before 60,000 signals were analysed.

3. Statistics

The data are given as mean value±SEM. Continuous variables were tested for normal segregation with the Kolmogorov-Smirnov-test. A statistical analysis was performed using a T-test for the comparison of two treated groups (analysis of variance: Levene-test) and by the single-sided ANOVA-analysis for several groups, followed by post hoc-analysis with LSD-correction. Category variables were compared with the aid of Fischer's-exact-test. A linear regression analysis and non-parametric two-dimensional correlations (Spearman rank correlation coefficient [rs]) were used in order to correlate the number of circulating apoptotic ECs with different cardiovascular risk factors (CVRF). Statistic significance was assumed, if the null-hypothesis could be rejected at p=0.05. All statistical analyses were performed with the aid of SPSS for Windows 9.0 (SPSS, Inc.).

### Example 1

In order to determine the number of circulating apoptotic ECs, FACS-analyses in patients with stable angina (SA, n=29), unstable angina (UA, n=14) or myocardial infarction (MI, n=13) were done, and compared with 41 healthy control persons matching with respect to age and gender. Circulating apoptotic ECs were defined as cells in which a detection of the endothelial marker proteins CD146 and vWF was positive, and annexin V-binding occurred. In addition, exclusively CD45⁻-cells were included in the analysis, in order to exclude potentially contaminating leukocytes. A representative analysis is shown in FIG. 1.

When compared to healthy control persons or to patients with stable CAD, patients in acute phases of CAD, defined as ACS (unstable angina and myocardial infarction, n=27), showed significantly higher concentrations of circulating apoptotic ECs (FIG. 2A). In addition, the concentrations of apoptotic ECs in patients with stable CAD were twofold higher, compared to healthy controls (FIG. 2A).

Earlier studies have already shown that the total amount of circulating ECs in ACS is increased (Hladovec J., Prerovsky I., Stanek V., Fabian J. Klin. Wochenschr. 1978; 56: 1033-1036; Mutin M., Canavy I., Blann A., Bory M., Shampol J., Dignat-George F. Blood. 1999; 93: 2951-2958). Therefore, in addition the ratio of apoptotic ECs to the total amount of circulating cells was quantified in order to exclude that the increase of total circulating ECs would wrongly increase the number of apoptotic ECs. Nevertheless, the portion of apoptotic ECs of the total amount of ECs was also significantly increased in patients with unstable angina and myocardial infarction (FIG. 2B).

The increase of apoptotic ECs in patients with ACS could also be shown, when a different set of endothelial markers, namely a receptor for vascular endothelial growth factor (VEGF), VEGF-receptor-1 (KDR), and vWF was used for a double staining. Although the overall number of apoptotic ECs was lower when KDR and vWF were used as EC-markers, even in this case patients with unstable angina and myocardial infarction exhibited significantly higher concentrations of circulating apoptotic ECs, compared to healthy control persons (UA/MI: 0.78±0.20/μl vs. control: 0.37±0.05/μl; p<0.005). Correspondingly, patients with CAD not only exhibited profoundly increased concentrations of circulating apoptotic ECs, but the number of circulating apoptotic ECs also correlated with the activity of the disease, as demonstrated by the significant increase in patients with ACS.

### Example 2

Furthermore, the influence of risk factors for an atherogenesis, namely age, gender, hypertension, diabetes, smoking, a positive family history regarding CAD as well as hypercholesterolemia, on circulating apoptotic ECs was examined. Of these generally established cardiovascular risk factors, a simultaneously detected disease of diabetes mellitus correlated significantly with an increased number of circulating apoptotic ECs (Table 3, below). In addition, increased concentrations of serum lipid (LDL-Cholesterin) were associated with an elevated EC-apoptosis, whereas a high HDL/LDL-ratio was found to be inversely associated with the concentration of apoptotic ECs (Table 3). It is interesting to note that a clinical manifestation of a coronary arterial disease (controls vs. stable angina vs. ACS) was profoundly associated with an increase of circulating apoptotic ECs (Table 3). Similar results were obtained, when the ratio of apoptotic ECs to the overall number of ECs was correlated with the above described factors (not depicted).

TABLE 3

| Factor | r, two-sided | N | p Spearman-Ranks |
|---|---|---|---|
| Age | 0.105 | 97 | 0.304 |
| Gender (m/f) | 0.011 | 97 | 0.916 |
| HTN | 0.051 | 56 | 0.712 |
| Diabetes mellitus | 0.357 | 56 | 0.007 |
| Nicotine | −0.075 | 56 | 0.581 |
| Family history | −0.077 | 56 | 0.573 |
| LDL-cholesterol | 0.222 | 80 | 0.048 |
| HDL/LDL-ratio | 0.282 | 80 | 0.011 |
| CRP | 0.241 | 84 | 0.027 |
| UA/MI vs. SA vs. | 0.496 | 97 | <0.001 |
| contr. | | | |

Recent studies have established markers of a low-grade inflammation, such as C-reactive protein (CRP) or serum amyloid A, as important prognostic parameters for the progression of an arteriosclerotic disease, both in healthy test persons as well as in patients with proven CAD (Blake G. J., Ridker P. M. Circ. Res. 2001; 89: 763-771). Therefore, the influence of the CRP-levels on the apoptosis of ECs was examined. The CRP-concentrations in serum correlated significantly with an elevated EC-apoptosis (Table 2). It is also of interest that the CRP-concentrations, measured with a highly sensitive assay, showed a profoundly significant correlation with the occurrence of apoptotic ECs even in the healthy control persons (r=0.495; p<0.007).

### Example 3

A therapy with statines is established both for the primary as well as the secondary prevention of a coronary disease in order to reduce cardiovascular events, indicating that statines reduce the activity of an arteriosclerotic disease (Maron D. J., Fazio S., Linton M. F. Circulation, 2000, 101, p. 207-213). Thus, it was prospectively examined whether a short-term treatment with atorvastatin (20 mg/day, four weeks) reduces the EC-apoptosis in patients with stable CAD. FIG. 3 shows that the treatment with atorvastatin significantly reduced both the number of circulating apoptotic ECs as well as their proportion of the total number of circulating ECs. The statin therapy also significantly reduced the concentration of LDL-cholesterol (p<0.02).

### Example 4

It was furthermore examined, whether an elevated apoptosis of CD45⁻ mature ECs could be associated with increased concentrations of endothelial precursor cells in peripheral blood. Circulating endothelial precursor cells were examined for the endothelial marker protein KDR and the EC-precursor-marker AC133 by immunocytochemical staining as described earlier, and analysed (Vasa M., Fichtlscherer S., Aicher A., Adler K., Urbich C., Martin H., Zeiher A. M., Dimmeler S. Circ. Res. 2001; 89: E1-7). It could be shown that increased concentrations of apoptotic endothelial cells were significantly associated with an increase of AC133⁺/KDR⁺ cells (FIG. 4). A similar correlation was observed, when the endothelial precursor cells were defined as CD34⁺/KDR⁺-cells (not shown).

1. A method for the identification and/or quantification of endothelial cells being related to cardiovascular diseases in a sample, wherein the method comprises the following steps:
(a) obtaining a sample to be analyzed containing endothelial cells;
(b) incubating the sample with one or several molecules that specifically bind to one or several of the following marker molecules of the endothelial cells:
endothelial cell-markers and/or markers for apoptosis, or
endothelial cell-markers and/or markers of endothelial precursor cells;
(c) identification and/or quantification of the endothelial cells on the basis of the bound molecules by using immunocytological methods; and
(d) comparing the result obtained for the sample to be analyzed with the result of a reference sample.

2. The method according to claim 1, wherein said endothelial cells are derived from a mammal.

3. The method according to claim 1, wherein said endothelial cells are selected from the group consisting of apoptotic endothelial cells, endothelial precursor cells, and mature endothelial cells.

4. The method according to claim 1, wherein said sample to be analyzed is selected from the group consisting of peripheral blood, cell culture-suspensions and suspensions containing cells that have been released mechanically, chemically and/or enzymatically from the wall of a vessel.

5. The method according to claim 4, wherein said sample to be analyzed is peripheral blood.

6. The method according to claim 5, wherein a coagulation inhibitor, is added to the peripheral blood.

7. The method according to claim 1, wherein said marker-binding molecules are selected from the group consisting of antibodies or parts or fragments thereof, and receptor ligands or parts thereof.

8. The method according to claim 7, wherein said antibodies or parts or fragments thereof comprise polyclonal antibodies, monoclonal antibodies, Fab-fragments, scFv-fragments, and diabodies.

9. The method according to claim 1, wherein said marker-binding molecules are present in solution or matrix-immobilized.

10. The method according to claim 1, wherein said marker-binding molecules are coupled to one or several detection molecules from the group consisting of fluorescein thioisocyanate, phycoerythrine, enzymes, and magnetic beads.

11. The method according to claim 1, wherein said marker-binding molecules are detected with an antibody being coupled to one or several detection molecules.

12. The method according to claim 1, wherein said endothelial cellular marker is selected from the group consisting of CD146, von Willebrandt-factor (vWF), and vascular endothelial growth factor-receptor 1 (VEGF-receptor-1).

13. The method according to claim 1, wherein said marker for apoptosis is selected from the group consisting of annexin V and PD-ECGF.

14. The method according to claim 1, wherein said markers of endothelial precursor cells are selected from the group consisting of CD133 and CD34.

15. The method according to claim 1, wherein furthermore at least one marker being characteristic for non-endothelial cells is determined.

16. The method according to claim 1, wherein said immunocytological methods are selected from the group consisting of flow cytometry and solid-phase-immunoassays.

17. The method according to claim 1, wherein said reference sample is derived from a mammal, wherein a cardiovascular disease was excluded.

18. The method according to claim 1, wherein said result for apoptotic endothelial cells is brought in relation with the result for the totality of endothelial cells.

19. The method according to claim 1, wherein said result for apoptotic endothelial cells is brought in relation with the result for the endothelial cells.

20. The method according to claim 1, further comprising a lysis of the erythrocytes between step (a) and (b).

21. The method according to claim 1, wherein said cardiovascular diseases are selected from the group consisting of stable and unstable angina, myocardial infarction, acute cardiac syndrome, coronary arterial disease and heart insufficiency.

22. A diagnostic kit, comprising means for performing a method for the identification and/or quantification of endothelial cells being related to cardiovascular diseases in a sample, wherein the method comprises the following steps:
(a) obtaining a sample to be analyzed containing endothelial cells;
(b) incubating the sample with one or several molecules that specifically bind to one or several of the following marker molecules of the endothelial cells:
endothelial cell-markers and/or markers for apoptosis, or
endothelial cell-markers and/or markers of endothelial precursor cells;
(c) identification and/or quantification of the endothelial cells on the basis of the bound molecules by using immunocytological methods; and
(d) comparing the result obtained for the sample to be analyzed with the result of a reference sample,
optionally together with additional components and/or excipients.

23. A method for the diagnosis and/or prognosis of cardiovascular diseases and/or for the monitoring of their therapy, wherein said method comprises the following steps:
(a) obtaining a sample to be analyzed containing endothelial cells;
(b) incubating the sample with one or several molecules that specifically bind to one or several of the following marker molecules of the endothelial cells:
endothelial cell-markers and/or markers for apoptosis, or
endothelial cell-markers and/or markers of endothelial precursor cells;
(c) identification and/or quantification of the endothelial cells on the basis of the bound molecules by using immunocytological methods; and
(d) comparing the result obtained for the sample to be analyzed with the result of a reference sample.

24. The method according to claim 23, wherein said therapy comprises the administration of lipid lowering substances, selected from the group consulting of statines, in particular atorvastain.

25. The method, according to claim 2, wherein said endothelial cells are derived from a human.

26. The method, according to claim 6, wherein said inhibitor is heparin.
